(19) United States
(12) Patent Application Publication
(10) Pub. No.: US 2009/0062612 A1
(43) Pub. Date: Mar. 5, 2009

(54) ENDOSCOPE SYSTEM

(75) Inventor: Tadaaki SUDA, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/194,883

(22) Filed: Aug. 20, 2008

(30) Foreign Application Priority Data
Aug. 27, 2007 (JP) 2007-219577

(51) Int. Cl. A61B 1/04

(52) U.S. Cl. 600/118

(57) ABSTRACT

An endoscope system comprises a scope, a first processor, and a first measuring device. The scope has an imaging sensor, a memory, and a reference signal output unit that outputs a reference signal instead of an image signal from the imaging sensor in a first operation for generating an analysis data. The first processor performs an image-processing operation of the image signal in a normal use, and performs the image-processing operation of the reference signal in the first operation. The first measuring device is connected to the first processor and measures a first characteristic of an image encoded by the reference signal on which the image-processing operation is carried out, in the first operation. The first processor transmits a measurement result by the first measuring device to the scope as the analysis data. The memory stores the analysis data when the measurement result is input to the first processor.

### BACKGROUND OF THE INVENTION

1. Field of the Invention

The present invention relates to an endoscope system, and in particular, to an endoscope system whose self-diagnostic operation is easily performed.

2. Description of the Related Art

An endoscope system that has a scope including an imaging sensor is proposed. When a problem occurs in the endoscope system, it is necessary to specify the context of the problem in the endoscope system. Such a problem may include not only a failure of at least one part of the endoscope system, but also an unintended performance of the endoscope system. For example, the case in which an image color does not match the user's intention is cited.

Japanese unexamined patent publication (KOKAI) No. 2004-261612 discloses an endoscope system that has a diagnostic device that performs an error check on each part of the circuit of the image-processing apparatus (the processor).

This diagnostic device can determine the failure of the endoscope system, however, it can not determine the context of the unintended performance of the endoscope system. Therefore, it is necessary to bring back the endoscope system from the health-care facility where the endoscope system is installed and used, to the service facility of the endoscope manufacturer, in order to determine the context of the problem including the unintended performance of the endoscope system. The processor that is installed at the health-care facility is a large apparatus and not very portable.

### SUMMARY OF THE INVENTION

Therefore, an object of the present invention is to provide an endoscope system for which a problem can be analyzed using the scope that is brought back, without bringing back the processor.

According to the present invention, an endoscope system comprises a scope, a first processor, and a first measuring device. The scope has an imaging sensor, a memory, and a reference signal output unit that outputs a reference signal instead of an image signal from the imaging sensor in a first operation for generating an analysis data. The first processor performs an image-processing operation of the image signal in a normal use, and performs the image-processing operation of the reference signal in the first operation. The first measuring device is connected to the first processor and measures a first characteristic of an image encoded by the reference signal on which the image-processing operation is carried out, in the first operation. The first processor transmits a measurement result by the first measuring device to the scope as the analysis data. The memory stores the analysis data when the measurement result is input to the first processor.

### BRIEF DESCRIPTION OF THE DRAWINGS

The objects and advantages of the present invention will be better understood from the following description, with reference to the accompanying drawings in which:

FIG. 1 is a construction diagram of the endoscope system in the embodiment;

FIG. 2 is a construction diagram of the detail of the scope;

FIG. 3 is a flowchart that shows a first operation for generating the analysis data; and

FIG. 4 is a flowchart that shows a second operation for preparing the diagnosis of the endoscope system.

### DESCRIPTION OF THE PREFERRED EMBODIMENTS

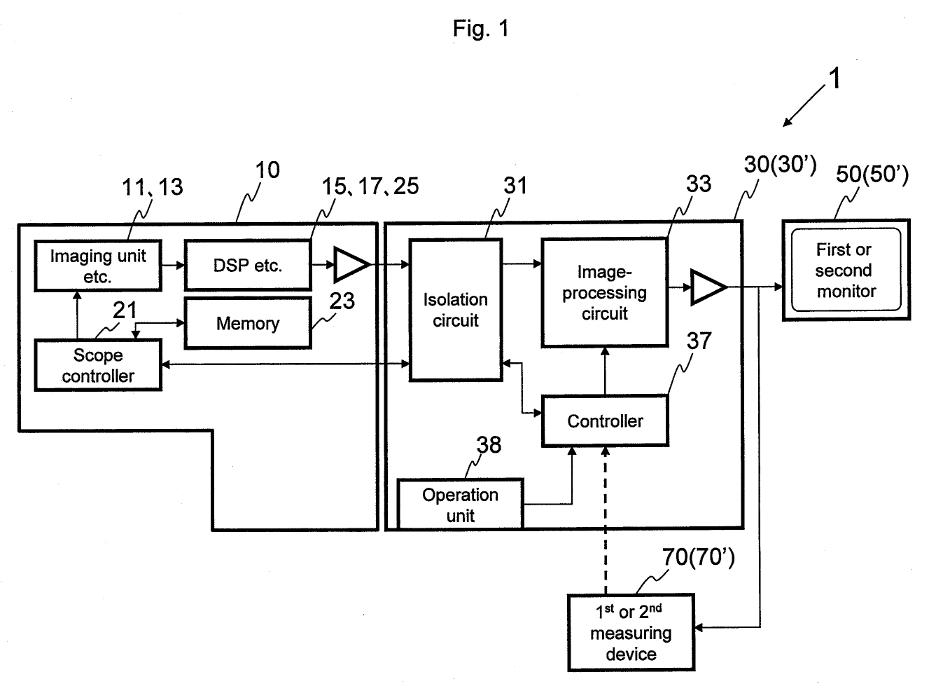

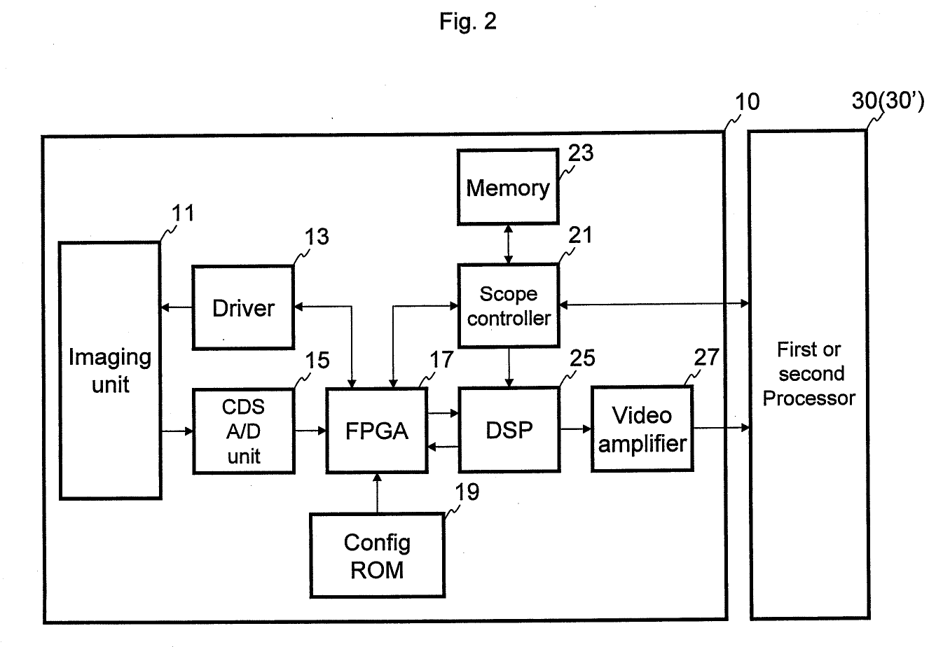

The present invention is described below with reference to the embodiment shown in the drawings. As shown in FIGS. 1 and 2, an endoscope system 1 in the embodiment comprises a scope 10, a first processor 30, and a first monitor 50, in a normal use mode in which the endoscope system 1 is normally used.

The scope 10, the first processor 30, and the first monitor 50 are installed at a health-care facility. In the normal use mode, a first measuring device 70 depicted in FIG. 1 is not necessary.

In a first operation for generating an analysis data that is used for a diagnosis of the endoscope system 1, the endoscope system 1 comprises the scope 10, the first processor 30, the first monitor 50, and a first measuring device 70.

The analysis data includes a characteristic of the image encoded by an image signal on which an image-processing operation in a first combination between the scope 10 and the first processor 30 is carried out. The characteristic of the image includes hue, saturation, lightness, gamma, halation, etc. The image-processing operation includes a primary image-processing operation by the scope 10 and a secondary image-processing operation by the first processor 30.

The first measuring device 70 is prepared at the health-care facility and connected to the processor 30 in the first operation for generating the analysis data.

In a second operation for preparing the diagnosis of the endoscope system 1, the endoscope system 1 comprises the scope 10, a second processor 30′, a second monitor 50′, and either the first measuring device 70 or a second measuring device 70′.

In the diagnosis of the endoscope system 1, the endoscope system 1 also comprises the scope 10, the second processor 30′, the second monitor 50′, and either the first measuring device 70 or the second measuring device 70′.

The second operation for preparing the diagnosis of the endoscope system 1 is also performed before the diagnosis of the endoscope system 1 is performed.

The second operation is an adjustment that matches a first color reproducibility in the first combination between the scope 10 and the first processor 30 and a second color reproducibility in a second combination between the scope 10 and the second processor 30′. In other words, by the second operation, the characteristic obtained by the image-processing operation in the second combination installed at the service facility is matched to the characteristic obtained by the image-processing operation in the first combination installed at the health-care facility.

In the embodiment, the health-care facility is a place, such as a hospital, etc., where the first processor 30, etc., are installed and the endoscope system 1 is normally used in the normal use mode.

The service facility is a place, such as a service station of the endoscope manufacturer, etc., where the maintenance of the endoscope system 1 is operated by the service personnel. Usually, the health-care facility is separate from the service facility.

The second processor 30′, the second monitor 50′, and the second measuring device 70′ are installed at a service facility. The scope 10 is brought back to the service facility from the health-care facility when it is repaired.

The second processor 30′ has the same function as the first processor 30. Similarly, the second monitor 50′ has the same function as the first monitor 50′.

It is desirable that the first measuring device 70 is used for the second operation and the diagnosis of the endoscope system 1. However, the second measuring device 70′ that has the same function as the first measuring device 70 may be used.

When the first measuring device 70 is used for the second operation and the diagnosis of the endoscope system 1, the first measuring device 70 is brought back from the health-care facility.

The scope 10 has an imaging unit 11 including an imaging sensor such as a CCD, etc., a driver 13 that drives the imaging unit 11, a CDS and ADC unit 15 that performs a correlated double-sampling operation and an A/D conversion operation, an FPGA (Field Programmable Gate Array) 17, a Config ROM 19 that stores various kinds of commands, a scope controller 21, a memory 23 that is non-volatile, a DSP (Digital Signal Processor) 25, and a video amplifier 27.

The first processor 30 has an isolation circuit 31, an image-processing circuit 33, a controller 37, and an operation unit 38. The second processor 30′ also has the same construction as the first processor 30.

The secondary image-processing operation by the first processor 30 is carried out on the image signal that is obtained by the scope 10, to generate the image displayed on the first monitor 50.

The first monitor 50 is connected to the first processor 30. The first monitor 50 is a display device that displays an image compatible with the predetermined video format.

Furthermore, another device, such as an external storage device that stores the data of the image signal, etc., and a printer that prints out the image encoded by the image signal may be connected to the first processor 30.

The first processor 30 and the second processor 30′ have a video signal output port. The image signal on which the image-processing operation is performed is output from the first processor 30 (the second processor 30′) to the first monitor 50 (the second monitor 50′), through the video signal output port.

In the normal use mode, the video signal output port of the first processor 30 is connected to the first monitor 50. Therefore, the image signal on which the image-processing operation is performed is output from the first processor 30 to the first monitor 50 through the video signal output port.

In the first operation for generating the analysis data, the video signal output port of the first processor 30 is connected to the first monitor 50 and the first measuring device 70. Therefore, the image signal on which the image-processing operation is performed is output from the first processor 30 to the first monitor 50 and the first measuring device 70 through the video signal output port.

In the second operation for preparing the diagnosis of the endoscope system 1, the video signal output port of the second processor 30′ is connected to the second monitor 50′ and either the first measuring device 70 or the second measuring device 70′. Therefore, the image signal on which the image-processing operation is performed is output from the second processor 30′ to the second monitor 50′ and the first measuring device 70 (the second measuring device 70′) through the video signal output port.

The first measuring device 70 is an analyzing device, such as a vector scope, a waveform monitor, a PC, etc., that measures the characteristic of the image encoded by a reference signal on which the image-processing operation in the first combination or the second combination is performed, and that displays the measurement result itself.

The measurement result is used for the first operation and the second operation that are described later.

Similarly, the second measuring device 70′ is also an analyzing device that measures the characteristic of the image encoded by a reference signal on which the image-processing operation in the second combination is performed, and that displays the measurement result itself.

Next, the details of each part of the endoscope system 1 are explained.

The imaging sensor of the imaging unit 11 receives the reflected light (or the exciting light) from the photographic subject through the objective optical system (not depicted).

The optical image of the photographic subject on the incident surface of the imaging sensor is captured, the photoelectric conversion is performed, and then the image signal encoding the optical image is output.

In the normal use mode, the image signal from the imaging unit 11 is output to the DSP 25 through the CDS and ADC unit 15 and the FPGA 17. The primary image-processing operation including a YC separation, etc., on the image signal output from the imaging unit 11 is carried out by the DSP 25.

Then, the image signal after the primary image-processing operation is amplified by the video amplifier 27 and output to the image-processing circuit 33 through the isolation circuit 31.

The secondary image-processing operation on the image signal output from the scope 10 is carried out by the image-processing circuit 33 so that a video signal is generated for image display on the first monitor 50.

In the first operation for generating the analysis data, the controller 37 of the first processor 30 transmits a first command corresponding to a color data collection mode to the scope controller 21.

The scope controller 21 transmits a mode-change command to the FPGA 17, after the scope controller 21 receives the first command. When the mode of the endoscope system 1 is changed from the normal use mode to the color data collection mode for the first operation, the FPGA 17 terminates the input of the image signal from the CDS and ADC unit 15 and outputs the reference signal that is previously encoded in the FPGA 17 to the DSP 25 instead of the image signal. The reference signal is output in time with the driving signal of the imaging sensor of the imaging unit 11.

After the completion of the output of the reference signal, the FPGA 17 transmits a first notice of the completion of output of the reference signal to the scope controller 21.

As the reference signal output unit, the FPGA 17 separately outputs a first reference signal regarding red, a second one regarding green, a third one regarding blue, and the other signal for analysis, as the reference signal.

As example of the other signal for analysis, a first analysis signal, a second analysis signal, etc., are cited.

The luminance value of the first analysis signal varies linearly with time, such as a saw-tooth waveform, used for measuring gamma.

The luminance value of the second analysis signal exceeds white level, and is used for measuring a Y-limit function such as halation. The Y-limit function is a function in the image-processing operation that mandatorily limits the luminance value to the predetermined value when a luminance value higher than the white level is input.

The primary image-processing operation on the reference signal output from the FPGA 17 is carried out by the DSP 25.

Then, the reference signal after the primary image-processing operation is amplified by the video amplifier 27 and output to the image-processing circuit 33 through the isolation circuit 31.

The first notice of the completion of output of the reference signal, which is one of the first reference signal regarding red, the second reference signal regarding green, the third reference signal regarding blue, and the other signal for analysis, is transmitted from the FPGA 17 to the scope controller 21. Furthermore, the first notice is also transmitted to the controller 37 of the first processor 30 through the scope controller 21.

When the controller 37 receives the first notice, the first processor 30 is set to a state where the reference signal output from the scope 10, on which the primary image-processing operation is carried out, can be received, by the controller 37.

The secondary image-processing operation on the reference signal output from the scope 10 is carried out by the image-processing circuit 33. Then, the reference signal on which the secondary image-processing operation is carried out is output from the video signal output port to the first measuring device 70.

The first measuring device 70 measures the characteristic of the image encoded by the reference signal and by it self displays a first characteristic value as the measurement result.

The user of the endoscope system 1 or the service personnel confirms the first characteristic value displayed on the first measuring device 70 and then manually input the first characteristic value to the first processor 30 using the operation unit 38 of the first processor 30. The first characteristic value is transmitted to and stored in the memory 23 through the controller 37, the isolation circuit 31, and the scope controller 21.

However, the first measuring device 70 may automatically input the first characteristic value to the first processor 30.

This above-mentioned process for each of the first reference signal, the second reference signal, the third reference signal, and the other signal for analysis is performed in series. The first characteristic value for all of them is stored in the memory 23, as the analysis data.

After storing the analysis data for all of the reference signals to the memory 23, a second notice of the completion of the measurement is transmitted from the scope 10 to the first processor 30 signaling that the first operation for generating the analysis data is finished.

After the first operation for generating the analysis data is finished, only the scope 10 is brought back from the health-care facility to the service facility, and the diagnosis of the endoscope system 1 is performed at the service facility. Therefore, the first processor 30 and the first monitor 50 may remain at the health-care facility.

Before the diagnosis of the endoscope system 1, the second operation for preparing the diagnosis of the endoscope system 1 is performed, consisting of an adjustment that matches the first color reproducibility in the first combination at the health-care facility and the second color reproducibility in the second combination at the service facility.

With the second operation, the characteristic of the secondary image-processing operation by the second processor 30′ will closely match the characteristic of the secondary image-processing operation by the first processor 30.

In the second operation for preparing the diagnosis of the endoscope system 1, the scope 10 including the memory 23 in which the measurement result by the first measuring device 70 is stored, the second processor 30′, the second monitor 50′, and the first measuring device 70 (or the second measuring device 70′) are used.

The second operation for preparing the diagnosis of the endoscope system 1 is operated at the service facility separated from the health-care facility. In the embodiment, it is not necessary to bring back the first processor 30, which is a large apparatus and not very portable, from the health-care facility to the service facility. Only the scope 10, which is a relatively portable device, is brought back from the health-care facility to the service facility.

In a manner similar to the first operation for generating the analysis data, in the second operation for preparing the diagnosis of the endoscope system 1, the reference signal is also output from the FPGA 17 and the first measuring device 70 (or the second measuring device 70′) also measures the characteristic of the image encoded by a reference signal on which the image-processing operation in the second combination is carried out.

However, in the second operation, the reference signal is output on the basis of a second command corresponding to an analysis mode that is different from the color data collection mode to the scope controller 21.

When the controller 37 of the second processor 30 transmits the second command to the scope controller 21, the reference signal is output from the FPGA 17 and the analysis data is also read out from the memory 23.

A second characteristic value in the measurement result in the second operation for preparing the diagnosis of the endoscope system 1 is displayed on the first measuring device 70 (or the second measuring device 70′).

The first characteristic value in the analysis data that is read out from the memory 23 is displayed on an output device, such as the second monitor 50′ or the first measuring device 70 (or the second measuring device 70′) etc.

The service personnel confirm the first characteristic value displayed on the second monitor 50′, etc., and manually adjust a setting value of the secondary image-processing operation in the second processor 30′ using the operation unit 38 so as to match the second characteristic value in the measurement result in the second operation for preparing the diagnosis of the endoscope system 1 (which may vary), to the first characteristic value (which is the fixed).

The setting value of the secondary image-processing operation includes a parameter regarding a color setting, such as brightness, etc.

When the adjustment of the setting value of the image-processing operation for all of the first reference signal, the second reference signal, the third reference signal, and the other reference signal, is complete, the second color reproducibility in the second combination between the scope 10 and the second processor 30′ should be closely matched to the first color reproducibility in the first combination between the scope 10 and the first processor 30.

After the adjustment, the diagnosis of the endoscope system 1 is performed by the service personnel.

In the embodiment, only the scope 10 is brought back from the health-care facility and the first processor 30 remains at the health-care facility, and the diagnosis of the endoscope system 1 is performed using the second processor 30′ whose characteristic is matched to the characteristic of the first processor 30. Therefore, both the context of the problem of the scope 10, as well as the context of the problem of the first processor 30 can be specified. Potential problems include not only a failure of at least one part of the endoscope system 1, but also the unintended performance of the endoscope system 1. For example, the case in which an image color does not match the user's intention may arise. Specifically, a problem regarding fine color reproducibility, such as a red color tone of the image displayed on the first monitor 50, etc., can be analyzed.

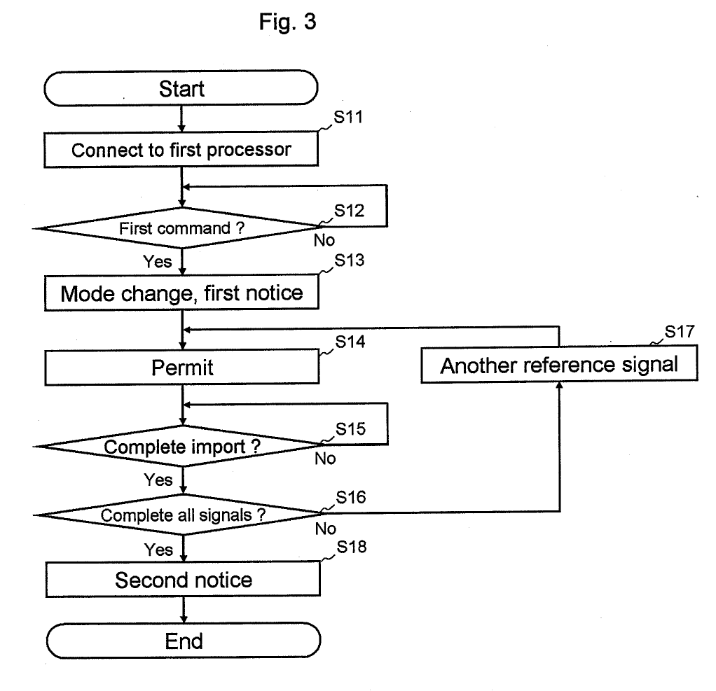

Next, the process of the first operation for generating the analysis data is explained using the flowchart in FIG. 3.

In step S11, the scope 10 is connected to the first processor 30. In step S12, it is determined whether the first command corresponding to the color data collection mode is transmitted from the controller 37 to the scope controller 21.

When it is determined that the first command is transmitted, in other words, when the endoscope system 1 is set to the color date collection mode by the user's or service personnel's operation, the operation continues to step S13. Otherwise, the operation in step S12 is repeated.

In step S13, the mode-change command based on the first command is transmitted from the scope controller 21 to the FPGA 17, so that the FPGA 17 terminates the input of the image signal from the CDS and ADC unit 15 and outputs the reference signal to the DSP 25.

After the output of the reference signal is complete, the first notice of the completion of output of the reference signal is transmitted from the scope 10 to the first processor 30.

In step S14, the first processor 30 permits an import of the reference signal output from the FPGA 17, in other words, the first processor 30 is set to a state where the reference signal output from the scope 10, on which the primary image-processing operation is carried out, can be received by the controller 37.

In step S15, it is determined whether the import of the reference signal is complete.

In the case that hue and saturation of the image encoded by the reference signal on which the image-processing operation is carried out are measured by the first measuring device 70, the measured values of hue and saturation are displayed on the first measuring device 70 as the first characteristic value. Then the first characteristic value is manually input to the first processor 30 using the operation unit 38, and the first characteristic value is stored in the memory 23 through the first processor 30 and the scope controller 21, and it is determined that the import of the reference signal is complete, the operation continues to step S16, otherwise, the operation in step S15 is repeated.

In step S16, it is determined whether the operation in steps S14 and S15 has been performed for all of the first reference signal, the second reference signal, the third reference signal, and the other reference signal.

When it is determined that the operation in steps S14 and S15 has been performed for all of the reference signals, in other words, when the first characteristic values regarding all of the reference signals are stored in the memory 23, the operation proceeds directly to step S18, otherwise, the operation continues to step S17.

In step S17, one of the reference signals, which has not been output yet, is output from the FPGA 17 so that the operation in steps S14 and S15 is performed.

In step S18, the second notice of the completion of the measurement is transmitted from the scope 10 to the first processor 30 so that the operation for generating the analysis data is finished.

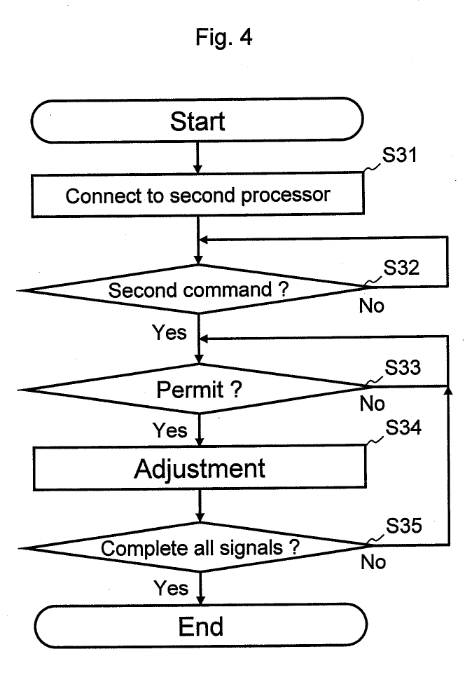

Next, the process of the second operation for preparing the diagnosis of the endoscope system 1 is explained using the flowchart in FIG. 4.

In step S31, the scope 10 that has the memory 23 in which the measurement result (the first characteristic values) is stored, the second processor 30′, and the first measuring device 70 (or the second measuring device 70′) are connected.

In step S32, it is determined whether the second command is transmitted from the controller 37 of the second processor 30′ to the scope controller 21.

When it is determined that the second command is transmitted, in other words, that the endoscope system 1 is set to the analysis mode operated by the service personnel, the operation continues to step S33, otherwise, the operation in step S32 is repeated.

Before the operation in step S33, the mode-change command based on the second command is transmitted from the scope controller 21 to the FPGA 17, so that the FPGA 17 terminates the input of the image signal from the CDS and ADC unit 15 and outputs the reference signal to the DSP 25. The reference signal is output in time with the driving signal of the imaging sensor of the imaging unit 11.

In step S33, it is determined whether the first notice is also transmitted from the scope 10 to the controller 37 of the second processor 30′ after the output of the reference signal so that the import of the reference signal output from the FPGA 17 is permitted by the second processor 30′. In other words, it is determined whether the second processor 30′ is set to the state where the reference signal output from the scope 10, on which the primary image-processing operation is carried out, can be received, by the controller 37.

When it is determined that the import is permitted, the operation is continues to step S34, otherwise, the operation in step S33 is repeated.

In step S34, the first measuring device 70 (or the second measuring device 70′) measures the characteristic of the image encoded by the reference signal output from the FPGA 17.

Furthermore, the analysis data is output from the memory 23. The first characteristic value in the analysis data which is read out from the memory 23 is displayed on the output device, such as the second monitor 50′, etc.

The second characteristic value in the measurement result in the second operation for preparing the diagnosis of the endoscope system 1 is displayed on the first measuring device 70 (or the second measuring device 70′).

The service personnel confirm the first characteristic value, (a fixed value), displayed on the second monitor 50′, etc., and manually adjust the setting value of the secondary image-processing operation in the second processor 30′ so as to match the second characteristic value, (which is variable), from the measurement result in the second operation, to the first characteristic value.

In step S34, it is determined whether the operation in steps S33 and S34 is performed for all of the first reference signal, the second reference signal, the third reference signal, and the other reference signal.

When it is determined that the operation in steps S33 and S34 has been performed for all of the reference signals, in other words, when the adjustments between the first characteristic value and the second characteristic value regarding all of the reference signals are complete, the operation is finished.

Thus, the endoscope system 1 is set to a state where the diagnosis of the endoscope system 1 regarding the characteristic including the color reproducibility of the first processor 30 can be performed even if the first processor 30 is not delivered to the service facility.

In the embodiment, numerical conversion of the characteristic of the image encoded by the reference signal is performed, and then the numerical converted characteristic value is stored in the memory 23 in the first operation for generating the analysis data for the diagnosis of the endoscope system 1.

Then the stored characteristic value is visually confirmed so that the second color reproducibility in the second combination between the scope 10 and the second processor 30′ at the service facility will be adjusted on the basis of the first color reproducibility in the first combination between the scope 10 and the first processor 30 at the health-care facility.

Therefore, the color reproducibility of the second processor 30′ at the service facility can be made to closely match that of the first processor 30 at the health-care facility. The analysis of the problem can be easily performed, in other words, the context of the problem can be easily specified, even if the first processor 30 is not transported from the health-care facility to the service facility.

Although the embodiment of the present invention has been described herein with reference to the accompanying drawings, obviously many modifications and changes may be made by those skilled in this art without departing from the scope of the invention.

The present disclosure relates to subject matter contained in Japanese Patent Application No. 2007-219577 (filed on Aug. 27, 2007) which is expressly incorporated herein by reference, in its entirety.

1. An endoscope system comprising:
a scope that has an imaging sensor, a memory, and a reference signal output unit that outputs a reference signal instead of an image signal from said imaging sensor in a first operation for generating an analysis data;
a first processor that performs an image-processing operation of said image signal in a normal use, and that performs said image-processing operation of said reference signal in said first operation; and
a first measuring device that is connected to said first processor and that measures a first characteristic of an image encoded by said reference signal on which said image-processing operation is carried out, in said first operation;
said first processor transmitting a measurement result by said first measuring device to said scope as said analysis data and said memory storing said analysis data when said measurement result is input to said first processor.

2. The endoscope system according to claim 1, wherein said reference signal output unit outputs said reference signal, in a second operation for preparing a diagnosis of said endoscope system before said diagnosis, said second operation being performed after said first operation and being performed with said scope is connected to a second processor that has a same function as said first processor;
said second processor performs said image-processing operation of said reference signal in said second operation;
either said first measuring device or a second measuring device that has a same function as said first measuring device, is connected to said second processor, and measures a second characteristic of an image encoded by said reference signal on which said image-processing operation is carried out in said second operation; and
said second processor has an operation unit that is configured to adjust a setting value of said image-processing operation of said second processor, so as to match said second characteristic to said first characteristic.

3. The endoscope system according to claim 1, wherein said reference signal output unit separately outputs a first reference signal regarding red, a second reference signal regarding green, a third reference signal regarding blue, and the other signal for analysis, as said reference signal.
